SEQUENCE LISTING (14 sequences)

SEQ ID NO: 1 - DNA, 27 bp, Homo sapiens
ctagcaaccatgaagtgggtggaatca

SEQ ID NO: 2 - DNA, 29 bp, Homo sapiens
cttggcagcatttctccaacaggcctgag

SEQ ID NO: 3 - DNA, 26 bp, Homo sapiens
accctggtgttggccagtgctgcact

SEQ ID NO: 4 - DNA, 28 bp, Homo sapiens
tcttgcttcatcgtttgcagcgctacac

SEQ ID NO: 5 - DNA, 21 bp, Homo sapiens
tgcgtttctcgttgcttacac

SEQ ID NO: 6 - DNA, 18 bp, Homo sapiens
atcagctccgacgaggtc

SEQ ID NO: 7 - DNA, 22 bp, Homo sapiens
actccagtaaaccctggtgttg

SEQ ID NO: 8 - DNA, 24 bp, Homo sapiens
ctgagcttggcacagatccttatg

SEQ ID NO: 9 - DNA, 22 bp, Homo sapiens
actccagtaaaccctggtgttg

SEQ ID NO: 10 - DNA, 25 bp, Homo sapiens
cagcaaagcagacttcctgttcctg

SEQ ID NO: 11 - DNA, 23 bp, Homo sapiens
acatccaggagagccaagcattg

SEQ ID NO: 12 - DNA, 25 bp, Homo sapiens
cagcaaagcagacttcctgttcctg

SEQ ID NO: 13 - DNA, 27 bp, Homo sapiens
ctggcaaccatgaagtgggtggaatca

SEQ ID NO: 14 - DNA, 24 bp, Homo sapiens
ctgagcttggcacagatccttatg

(19) United States
(12) Patent Application Publication
(10) Pub. No.: US 2016/0058896 A1
(43) Pub. Date: Mar. 3, 2016

(54) METHOD AND COMPOUND FOR TREATMENT OF CANCER USING PHOSPHOROUS-32 LABELED DNA

(71) Applicants: Yanping Kong, Merrimack, NH (US); Jinhong Liu, Merrimack, NH (US)

(72) Inventors: Yanping Kong, Merrimack, NH (US); Jinhong Liu, Merrimack, NH (US)

(21) Appl. No.: 14/822,841

(22) Filed: Aug. 10, 2015

Related U.S. Application Data
(63) Continuation-in-part of application No. 14/172,819, filed on Feb. 4, 2014.
Application No. 14/172,819 is a continuation-in-part of application No. 12/886,127, filed on Sep. 20, 2010, now Pat. No. 8,642,007.

(51) Int. Cl. A61K 51/04

(52) U.S. Cl. CPC A61K 51/0497; A61K 51/0491

(57) ABSTRACT

This invention provides a combination of a DNA strand/fragment and isotope therapy that is applied to a cancerous tissue to selectively kill cancer cells with minimal negative effects on surrounding non-cancerous cells. The mechanism of cancer cells uptake of large DNA fragment (larger than 30 bp) is endocytosis which does not occur in normal organ cells. We are the first to identify this difference and use the selectivity to deliver isotope P-32 to treat cancer. The DNA fragments with labeled isotope are able to be absorbed by the tumor cells and bind the tumor cell's DNA through recombination, and then the isotope kills the tumor cells. Illustratively, a gene or a DNA fragment is employed as a carrier to deliver the P-32 which can kill cancer cells through radioactive emission. Appropriate doses are provided to patients as part of a medical treatment method.

### RELATED APPLICATION

This application is a continuation-in-part of co-pending U.S. patent application Ser. No. 14/172,819, filed Feb. 4, 2014, entitled METHOD AND COMPOUND FOR TREATMENT OF CANCER USING PHOSPHOROUS-32 LABELED DNA, which is a continuation-in-part of co-pending U.S. patent application Ser. No. 12/886,127, filed Sep. 20, 2010, entitled METHOD AND COMPOUND FOR TREATMENT OF CANCER USING PHOSPHOROUS-32 LABELED DNA, now U.S. Pat. No. 8,642,007, issued Feb. 4, 2014, the entire disclosure of each of which applications is herein incorporated by reference.

### FIELD OF THE INVENTION

This invention relates to cancer treatments and medications and more particularly to treatments and medications associated with gene therapy.

### BACKGROUND OF THE INVENTION

Cancer is an abnormality in a cell's internal regulatory mechanisms that results in uncontrolled growth and reproduction of the cell. Normal cells make up tissues, and when these cells lose their ability to behave as a specified, controlled, and coordinated unit, the defect leads to disarray among the cell population. When this occurs, a tumor begins to propagate.

In addressing a cancerous condition, the essence of many medical treatments and procedures involves the removal or destruction of the tumor tissue. Examples of significant types of treatments include the surgical removal of cancerous growths and the destruction of metastatic tumors through chemotherapy and/or radiation therapy.

Surgery often is the first step in the treatment of cancer. The objective of surgery varies. Sometimes it is used to remove as much of the evident cancerous tumor as possible, or at least to “debulk” it (remove the major bulk(s) of tumor. However, not all tumors are amenable to surgery. Some may be located in parts of the body that render them impossible to completely excise. Chemotherapy is another common form of cancer treatment. Essentially, it involves the use of medications which specifically attack rapidly dividing cells (such as those found in a tumor) throughout the body.

Unfortunately, other cells in the human body that also normally divide rapidly (such as the lining of the stomach and hair) also are affected by chemotherapy. For this reason, many chemotherapy agents induce undesirable side effects such as nausea, vomiting, anemia, hair loss or other symptoms. As also discussed generally above, radiation therapy is another commonly used weapon in the fight against cancer. Ionizing radiation kills cancer by penetrating skin and intervening tissue, and damaging the DNA within the tumor cells. The radiation is delivered in different ways. The most common delivery technique involves directing a beam of radiation at the patient in a highly precise manner, focusing on the tumor. A radioisotope can be safely used to deliver local radiation for cancer treatment. A typical example of a radioisotope is I-131 for the treatment of thyroid cancer.

Another radiation method sometimes employed, called brachytherapy, involves implanting radioactive pellets (seeds) or wires in the patient's body in the region of the tumor. Radiation is generally delivered to a very targeted area to gain local control over a cancer (as opposed to treating the whole body, as is accomplished using chemotherapy).

A number of other cancer therapies exist, although presently, the majority of such treatments are under exploration in clinical trials, and few has become a standard of care. Examples of such varied treatments include the use of immunotherapy, monoclonal antibodies, anti-angiogenesis factors and gene therapy. A brief description of each of these relatively new treatment regimes is as follows:

Immunotherapy: There are various techniques designed to assist the patient's own immune system fight the cancer, quite separately from radiation or chemotherapy. Oftentimes, to achieve the goal, researchers inject the patient with a specially derived vaccine that strengthens the particular immune response needed to resist the cancer.

Monoclonal Antibodies: These are antibodies designed to attach to cancerous cells (but not normal cells) by taking advantage of differences between cancerous and non-cancerous cells in their antigenic and/or other characteristics. The antibodies can be administered to the patient alone or conjugated to various cytotoxic compounds or in radioactive form, such that the antibody preferentially targets the cancerous cells, thereby delivering the toxic agent or radioactivity to the desired cells.

Anti-Angiogenesis Factors: As cancer cells rapidly divide and tumors grow, they can soon outgrow their blood supply. To compensate for this, some tumors secrete a substance believed to help induce the growth of blood vessels in their vicinity, thus providing the cancer cells with a vascular source of nutrients. Experimental therapies have been designed to arrest the growth of blood vessels to tumors, thereby depriving them of needed sustenance.

Gene Therapy: Cancer is the product of a series of mutations that ultimately lead to the production of a cancer cell and its excessive proliferation. Cancers can be treated by introducing genes to the cancer cells that will act either to check or stop the cancer's proliferation, turn on the cell's programmed cell mechanisms to destroy the cell, enhance immune recognition of the cell, or express a pro-drug that converts to a toxic metabolite or a cytokine that inhibits tumor growth.

Another option for treatment in certain types of cancers is to employ an isotope that is tailored to be taken-up by the particular organ or tissue. For example, Iodine 131 is employed to treat thyroid cancer. The thyroid cancer cells have hundreds more times the potential to attract in the radioactive Iodine I-131 than other cells. The Iodine isotope effectively kills cancer cells in the thyroid. Advantageously, the radioactive wave of I-131 does not travel far, so it does not kill the cells of other organs than thyroid tissue. This renders the administration of Iodine a safe treatment in thyroid cancer and over active thyroid disease, and it has been used in this context for decades.

It is therefore desirable to provide a compound/agent and treatment method employing such a compound/agent, which destroys, and hence either facilitates the removal of or inhibits the further growth of tumor cells and tissue, while exhibiting mainly local effects and minimal or no systemic toxicity. This compound and treatment method should accomplish its goals in a manner that is free of significant damage to non-cancerous cells and that is highly selective for cancer cells. The compound and treatment method should also potentially be applicable to a wide variety of organs and tissues.

### SUMMARY OF THE INVENTION

This invention overcomes the disadvantages of the prior art by providing a combination of a (typically) double strand DNA fragment and isotope therapy that is applied to a cancerous tissue to selectively kill that associated cancer cells with minimal negative effects on surrounding non-cancerous cells. Illustratively, the compound/agent and associated treatment method combines molecular biology and nuclear medicine to provide an effective agent that selectively attacks cancerous tumors in a wide variety of organs and tissues.

Functionally, the specific DNA fragments with labeled isotope are able to bind the tumor cells DNA, and then the isotope kills the tumor cells. A DNA fragment is employed as a carrier to deliver the P-32 which can kill cancer cells through radioactive emission. Unlike traditional gene therapy, which employs a gene to express a protein, which can suppress the cancer cell growth or increase the sensitivity for radiation therapy or chemotherapy, the illustrative embodiment actually binds the radioactive substance via a DNA fragment. The illustrative embodiment produces the compound/agent containing a DNA fragment and P-32 through use of conventional P-32 labeling techniques such as those employed in molecular biology experiments (for example experiments used to test gene expression and gene amplification potency). In the illustrative embodiment, however, the same P-32 labeled DNA is employed directly for cancer treatment through a novel medical treatment method.

In an illustrative embodiment, the compound/agent is synthesized using conventional P-32 labeling techniques and an associated commercially available labeling kit that binds P-32 to a DNAfragment appropriate to migrate into, and bind with, DNA of a tumor cell via recombination or another mechanism. Illustratively, the AFP gene is used because it is associated with certain types of tumor cells (in the liver, for example), such as the Huh7 cell types. However, in further embodiments, other DNA fragments, including different genes or sequences, tumor DNAs can also be used. An appropriate fragment length of the AFP gene (or other (typically) DNA) is labeled with P-32. The fragment length is highly variable, amounting to between approximately 30 base pairs (bp) to approximately 10 kbp in various embodiments. A plurality of different fragment lengths can also be combined in the compound/agent. In one embodiment of human treatment method an illustrative fragment length of 30 bp to 10 kbp can be used. However, in certain protocols, the range of fragment lengths can be more closely defined. In a human treatment method, the tumor is initially imaged to determine tumor size and characteristics. An initial dose of 1-160 mci (of radioactivity) is then administered depending upon tumor size and patient age and weight. The patient is observed to determine whether sufficient compound/agent has been administered, and if not, more is administered. After administration (typically 1-3 months subsequent) the patient's tumor is imaged to determine the prognosis. If prognosis is less than optimal, one or more additional administrations of the compound/agent can be undertaken. In another embodiment, illustrative compound and method can also be used in diagnosis of cancer and associated conditions. After administering an appropriate dose of the P-32 labeled DNA, a whole body scan can be applied to the patient within approximately 24 to 72 hours. Based upon the radioactivity of the P-32, which binds to the genomic DNA in the affected cells, the cancerous region is clearly visible in the nuclear scan.

In alternate embodiments, where the P-32 labeled DNA is to be employed in the treatment and/or diagnosis of other types of cancer, it is expressly contemplated that a DNA fragment more-specific to the affected cells can be employed. For example if a cancer cell exhibits a different gene in elevated quantities, then a fragment with the ability to bind to that particular sequence can be employed. The labeling of this alternate fragment can occur in accordance with conventional labeling procedures in an illustrative embodiment.

In yet other alternate embodiments, the DNA for use in generating the treatment compound can be extracted from the patient's blood, body fluid or directly from the tumor. The DNA fragments used can be a group of fragments. The cancer undergoing treatment can be of any type suitable for treatment in addition to liver cancer. Also, the compound (DNA fragments labeled with P-32) generated herein for treatment of the cancer can be derived using a kit that includes appropriate instructions, protocols and/or reagents that facilitate the generation of the compound in a deliverable/infusible solution, then be injected to the patient through appropriate artery which supplies the tumor or through veins, body cavities or directly to tumors; and subsequent monitoring of results in relation to cancer cell death (or diagnosis).

### BRIEF DESCRIPTION OF THE DRAWINGS

The invention description below refers to the accompanying drawings, of which:

FIG. 1 is a photomicrographic diagram showing a dot-blot result associated with an arrangement of a plurality of Hep3B and Huh7 liver cells that have undergone treatment with a P-32 labeled compound/agent and depict various states of exposure based upon the treatment parameters, in which such liver cancer Huh7 cells are capable of significant dosage of the compounds without (free of) an associated transfection agent, and such is dose dependent;

FIG. 2 is a photographic diagram of excised tumors obtained from three groups of animal test subjects, including a control group, a group injected with P-32 only, and a group injected with P-32 AFP DNA compound/agent according to an illustrative embodiment; It demonstrated that the compound treated mice had smaller tumors and better survival;

FIG. 3 is a flow diagram of a human medical treatment procedure employing the P-32 AFP DNA compound/agent according to an illustrative embodiment;

FIG. 4 is a photographic diagram of a sample of cells to which a fluorescent-marked AFP-based compound in accordance with an embodiment herein has been applied, showing the cell membranes as darkened regions;

FIG. 5 is a photographic diagram showing a fluorescent image of the cell sample of FIG. 4, showing the presence of concentrations of the compound at the cell membranes;

FIG. 6 is a photographic diagram showing magnified view of a portion of the sample of FIG. 4;

FIG. 7 is a photographic diagram showing a fluorescent image of the cell sample of FIG. 6, showing the presence of concentrations of the compound at the cell membrane; and

FIG. 8 is a bar graph showing experimental results in which P-32 labeled DNA fragments in accordance with embodiments herein are taken in significant concentrations by the liver cancer cells (Huh7) at various times (i.e. as soon 10 minutes after incubation), compared to uptake of P-32 labeled DNA by normal liver cells (THEL3), which is relatively insignificant across the representative time span/intervals.

### DETAILED DESCRIPTION

A compound/agent and associated medical treatment method employing the compound/agent that selectively kills tumor cells using P-32 isotope carried on a DNA fragment is provided herein. Illustratively, the P-32 isotope is bound to a fragment of the alpha-fetoprotein (AFP) gene. In an illustrative embodiment, the term “fragment” is defined to include a sequence of contiguous base pairs (bp) ranging between approximately 30 bp to 2032 bp (the full length of the gene). The bonding of P-32 isotope is accomplished using conventional labeling techniques. The DNA fragment that is produced is employed as a carrier for P-32 isotope into cancer cells, so as to kill them through radiation emitted from the P-32 once the fragment is absorbed by the cell and bound to the cell's DNA. It should be understood by those of skill that the terms DNA “fragment” and “gene” herein typically refer to a strand (e.g. genomic) DNA sequence of bps, capable of maintaining stable within the bloodstream of a patient during the applicable treatment duration. In various alternate embodiments/implementations, a single strand P-32 labeled DNA fragment can be employed providing that it can remain stable during treatment, and uptake by, the affected cells.

### I. Production of the Compound/Agent

The compound/agent can be generated for use by conventional processes using a predetermined fragment of plurality of different fragments of the AFP gene. The sequence of this gene is well known. In an embodiment, the fragments can be produced using a conventional PCR or DNA synthesis machine to produce the p-32 AFP DNA fragments, the DNA length designed as 30-2032 bps. The DNA fragments can be any part of the whole AFP DNA sequence. In an embodiment in which the compound/agent is employed as a probe to determine cell-uptake, as described further below, 50 ng (or another quantity) of AFP cDNA is P-32[alpha-dCTP]-labeled using random primed DNA labeling kit. The labeling product is then purified to exclude the unincorporated nucleotides. In an exemplary embodiment, length of the resulting purified P-32 AFP DNA is at least 20 bp, but other lengths are expressly contemplated as described above. The compound also can be produced with a pair of designed primer to do PCR with P-32 alpha-dCTP using AFP DNA as template. Radioactivity of the compound can then be quantified by scintillation counter. The associated counts per minute (cpm) of the P-32 labeled probe is determined by the scintillation counter. 1 ul of labeled reaction can be used for quantification in an exemplary embodiment.

Note that the term “compound” as used herein refers to a combination consisting of various chemicals, solvents, agents and biologics (e.g. DNA fragments), which can be administered together, or in separate doses/packages at single time, or over a time span, to a patient or other subject for the purpose of treatment and/or diagnosis of a condition (e.g. cancer). Thus, the term “compound” should be taken broadly as used herein.

### II. Cell-Level Test of Compound/Agent Effectiveness

With a focus on liver cancer cells as an initial target, the goal of an initial test was to determine whether fragments of AFP DAN are capable of migrating into liver cancer cells, binding to DNA and remaining bound for sufficient time periods. The first test relates to whether AFP DNA fragments are capable of migrating into liver cancer cells efficiently.

Uptaking of 32p-labeled AFP DNA Fragments by Hep3B and Huh7 Hepatoma Cells:

A. Materials Employed
1. Cell lines: Hep3B and Huh7 hepatoma cell lines.
2. Radioactive materials: 32p[alpha-dCTP] (available from Perkin Elmer Life Sciences, Catalog No. BLU513H250uc).
3. Human alpha fetoprotein (AFP) plasmid (pCMV-sport6-AFP) (available from Open Biosystems, Catalog No. MHS1010-7430075)
4. Gel extraction kit (available from Qiagen: Catalog No. 20021)
5. Random primed DNA labeling kit (available from Roche USA, Catalog No.11004760001)
6. G-50m Micro Cloumns (available from GE Healthcare; Catalog No. 28903408)
7. Transfection agent: FuGene
8. DNA isolation kit, DNAzol (available from Invitrogen, Catalog No. 10503-027)

B. Methods Employed
1. Perform PCR purification of all lengths of AFP cDNA:
Using pCMV-sport6-AFP as the template, the whole length of AFP cDNA is PCR amplified by using the following pair of primers (P1, P2):

P1: CTAGCAACCATGAAGTGGGTGGAATCA;
P2: CTTGGCAGCATTTCTCCAACAGGCCTGAG

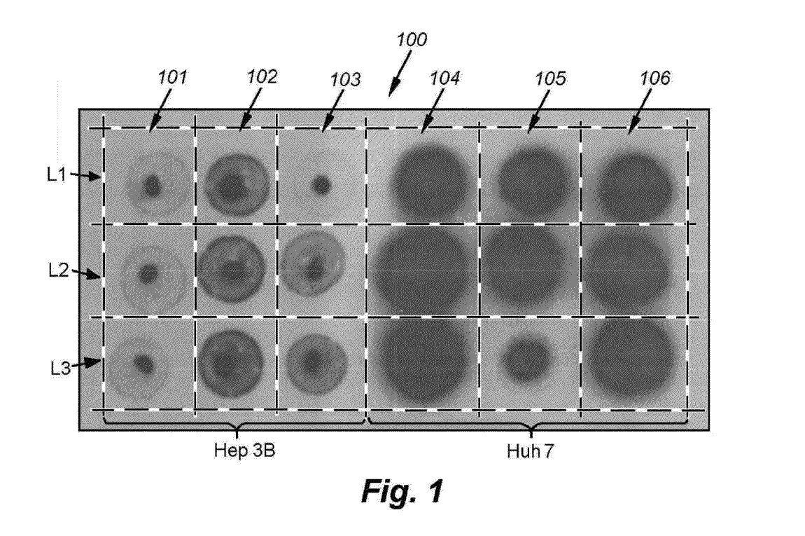

2. Preparation of 32P-labeled AFP probe (as described also above):
50 ng of AFP cDNA from step 1 is 32p[alpha-dCTP]-labeled using a random primed DNA labeling kit. The labeling product is purified to exclude the unincorporated nucleotide. The length of purified probe is at least 20 bp.
3. Radioactivity of probe is quantified by a scintillation counter, thereby determining the cpm of labeled probe. 1 ul of labeled reaction is used for quantification.
4. Cell preparation and treatment with radio-labeled AFP probe in which the exponentially growing hepatoma cells (Hep3B and Huh7) are trypsinized one day before treatment and seeded on 24-well plate at the density of 6×10⁴ cells per well. This well is not shown. However the arrangement is represented by the dot blot photomicrograph 100 in FIG. 1, where the genomic DNA from cells in each of the groups is arranged after treatment. The results from the treated cells are arranged in six lines (columns) 101-106. Columns 101-103 contain Hep3B cells and columns 104-106 contain Huh7 cells. There are three rows L1, L2 and L3 in the arrangement. Except as described below, the treatments are discretely provided to each of three cells (L1, L2 and L3) for each cell line (101-106):
Lines 101 and 104: 6 ul of 32p-labeled probe without adding any transfection reagent.
Lines 102 and 105: 6 ul of 32p-labeled probe with transfect ion agent following the proportion of 3 ul FuGene+2 ug 32P-labeled DNA. FuGene is a commercial regent which assists in allowing DNAs to migrate into cells.
Lines 103 and 106: 6 ul of radiolabeled probe with transfusion agent following the proportion of 8 ul FuGene+2ug 32P-labeled DNA.
The cell at location 105, L3 was provided with 1 ul of 32p-labeled probe as a comparison to all other cells, which were provided with 6 ul of 32p-labeled probe.
6. The cells are incubated with radioactive probe for 12-16 hours. The genomic DNAs are then isolated from the cells by DNAzol. The incorporated radioactive DNA fragments into the genome are thereafter quantified by a scintillation counter. In addition, whole amount of genomic DNA samples are dot-blotted on the nylon membrane, and the membrane is exposed to a Kodak X-film, which provides the cellular-level image shown in FIG. 1.

C. Result and Conclusions of Cellular-Level Treatment:

The arrangement 100 in FIG. 1 shows the results of treatment of individual Hep3B cells (lines 101-102) and individual Huh7 cells (lines 104-106) using the various treatment parameters described in Section B above. The following are specific results based upon the exposed image of each cell and the detected cpm value:
1. As indicated partially by the extreme darkness of the exposure. Huh7 hepatoma cells can uptake 32p-labeled AFP fragments, but Hep3B hepatoma cells appear to uptake a minimal amount of 32p-DNA fragments. The difference is approximately 66 times greater in Huh7 than Hep3B. Some promise may be shown in the treatment of Hep3B cells of line 102. There is no significant difference between lines 104, 105 and 106.
2. A lower dose of AFP DNA (1 ul rather than 6 ul) results in less uptake, as shown by the cell at line 105, L3. This difference is significant based upon a count of 302 cpm for the lower dose cell, versus 7400 cpm. This shows that the uptake of DNA by the Huh7 cell is dose (of DNA) related.
3. The AFP DNA fragments used can readily migrate into the cancer cells without using of a transfection agent, particularly in the case of Huh7.
4. The AFP DNA fragments remain within the cancer cell so as to provide a desired dosage of ionizing radiation, which can be detected by dot blot.
5. More generally, based upon the radioactivity readings (count as cpm) of genomic DNA isolated from hepatocarcinoma cells after incubation with P-32 labeled AFP DNA fragments, the Huh7 cells can uptake P32-labeled AFP DNAs, which is not affected by adding FuGene. Hep3B cells are minimally able, or unable to uptake the DNAs. The result is significant based upon a determined difference in counts 111.3 vs 7400 (e.g. 66 times difference).

### III. Live Animal Model Experiment: in vivo Treatment for Liver Cancer

Based upon proof that AFP DNA fragments can penetrate into liver cancer cells without (free-of) a transfection agent, a series of animal tests are performed on tumors. The following procedure steps are provided:
1. Prepare treatment compound/agent containing AFP DNA fragments and label the DNAs with P-32 isotope

A. Materials Employed
1. AFP vector (dilute the plasmid to final concentration 50 ng/μl by water)
2. AFP Forward primer P1 and P2 (20 μmol) in H₂O, Reverse prime (20 μmol) in H₂O (from Invitrogen):

P1 sequence (1521-1546):
ACCCTGGTGTTGGCCAGTGCTGCACT;
P2 sequence (1655-1682):
TCTTGCTTCATCGTTTGCAGCGCTACAC

3. DNA Labeling kit components:

| | |
|---|---|
| dATP | D4026A |
| dCTP | D4028A |
| dTTP | D4029A |
| dGTP | D4027A |

LA Taq DNA polymerase DRR02A (available from Takara Bio)
[α-P³²]dATP
[α-P³²]dCTP (available from Fu Rui Biotechnology, Beijing)
4. Illustra MicroSpin G-50 Columns (available from Amersham Pharmacia)
5. Mini-monitor (Morgen Series 900)
6. PCR thermal cycle (MJ Research PTC-200)

The above materials are handled and used in accordance with ordinary skill and the respective manufacturers' recommended procedures.

B. Compound/Agent Production Method Using Materials
1. Prepare 0.1 mmol/L dCTP, 0.1 mmol/L dATP by TE.
2. Prepare dNTP solution containing dTTP, dGTP each at 10 mmol/Lₒ
3. Set up amplification radiolabeling reactions for 10 time repetition;
   With each reaction containing:

| | |
|---|---|
| 10xLA buffer | 5 μl |
| 10 mmol/L dNTP | 1 μl |
| 0.1 mmol/LdCTP | 1 μl |
| 0.1 mmol/LdATP | 1 μl |
| Sense primer | 2 μl |
| Antisense primer | 2 μl |
| AFP template DNA | (50 ng/μl) 1 μl |
| [α--(32)P]dATP | 5 μl (10 μci/μl) |
| [α--(32)P]dCTP | 5 μl (10 μci/μl) |
| LA Taq DNA polymerase | 0.4 μl |
| H₂O | 26.6 μl. |

4. Gently tap the side of the reaction tube to mix ingredients.
5. Set up reaction following the following sequence of thermal parameters and associated exposure times:
   1) 94° C. 3 min
   2) 94° C. 30 s
   3) 55° C. 30 s
   4) 72 ° C. 5min
   Repeat step 5 from exposure times/settings (2) to (4) for 40 cycles
6. Remove the tubes from the thermal cycle (step 5). Then remove remaining unincorporated dNTPs with G-50 columns according to the manufacture's manual.
7. Next, employ the series 900, mini-monitor to measure the yield and the specific activity of radiolabeled AFP DNA fragments.

C. Animal Trials

Once the associated radioactivity activity of the DNA fragments resulting from the process has been determined, the compound/agent is prepared into injection into live animals experience liver cancer tumors. Before injection, the preparation of animals with liver tumors is the next step in the testing process.

In an example, H22 cells are injected subcutaneously at the flunk area of the receipt mice. Kunming nu/nu mice, male 22-24 gm are used in an example. At each injection site, 1×10⁶ of H22 liver cancer cells are injected subcutaneously into the nude mice at flunk area. The tumor nodules are noted 6 days after injection. The mice are divided into three groups: a control group, (A), which receives only an injection of normal saline in the same volume as other groups receiving the compound/agent (the number n of this group equals 11); a second group (B) receiving the P-32 isotope only 5 uci per mouse (n=10); and a group (C) that receives the P-32 labeled AFP DNA compound/agent 5 uci per mouse (n=8).

The saline, P-32 isotope and P-32 AFP DNA compound are each injected into the peritoneal cavity in mice for liver cancer treatment on the sixth day after H22 injection and notation of resulting tumors. Following injection of the cancer treatment, survival, tumor size and radioactivity in the tumor tissue were the endpoint outcome to study. In this trial, only a single injection is made.

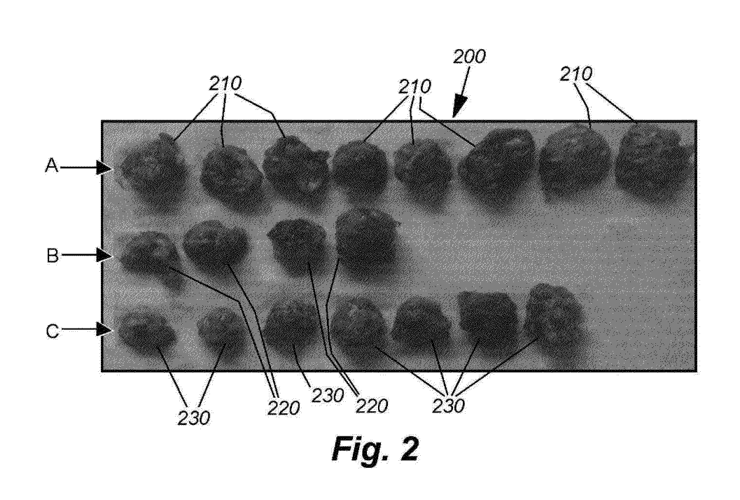

The subject animals are then observed for two weeks, and thereafter euthanized for subsequent tumor study. Tumors are removed from surviving animals and are shown in the photographic diagram 200 of FIG. 2. The extracted tumors for each of three groups A, B and C are displayed in corresponding photographic diagram rows A, B and C. The following table also exhibits the measured results:

| Animal | Number | | Tumor | Tumor Weight- | Radio- |
|---|---|---|---|---|---|
| Test | of | | Weight | to-Body | activity |
| Group | Deaths | Treatment | (grams) | Average (%) | (cpm) |
| Group A | 3 | Saline only | 8.75 | 16.8 | — |
| Group B | 6 | P-32 only | 7.2 | 15.2 | 41 |
| Group C | 1 | P-32 AFP | 5.47* | 12.23* | 123* |
| | | DNA | | | |

*P < 0.01

The results show higher radioactivity detected in group C mice which are treated with P-32 AFP DNA. This result suggests The P-32 labeled AFP DNA can bind the cancer cells' DNA and maintain in the cells longer, so as to kill the cells via radiation exposure. Only one mouse has died in Group C, but three have died in group A and six have died in Group B. This result shows the best survival in the Group treated with P-32 labeled AFP DNA, and the worst survival in the P-32 treated group. More specific results now follow below. This result suggests that P-32 labeled AFP DNA fragment treatment significantly improves survival in liver cancer.

Group A mice treated with normal saline, have tumors (210 in FIG. 2) that are harvested on the fourteenth day of treatment. The average tumor weight on the fourteenth day is 8.75 grams, in which the tumor weight to body weight ratio averages 16.8%.

Group B mice treated with normal P-32, have tumors (220 FIG. 2) that are harvested on the fourteenth day of treatment. The average tumor weight on the fourteenth day is 7.2 grams, in which the tumor weight to body weight ratio averages 15.2%.

Group C mice treated with P-32 AFP DNA compound/agent, have tumors that are harvested on day 14^{th} of treatment. The average tumor weight on the 14^{th} days is 5.47 grams, in which the tumor weight to body weight ratio averages 12.23%.

The table shows the relative radioactivity based on radioactive count from the tumor tissue on the 14^{th} day. Notably, the count in the Group C tumors is significantly higher than that of group B, indicating that the tumors retained the agent with radioactive P-32 more effectively.

More generally, as depicted visually in FIG. 2, tumors in group C at the end point (14 days) are noticeably smaller than those of Group A and B. Combined with the measured radioactivity results, which is significantly higher in Group C than in Group B, the results overall suggest that the P-32 AFP DNA fragments binds with the tumor cells DNA and maintains the P-32 within the cancer cells longer.

### IV. Medical Treatment Method for Human Liver Cancer Treatment with Phosphorous-32 Labeled AFP DNA Fragments

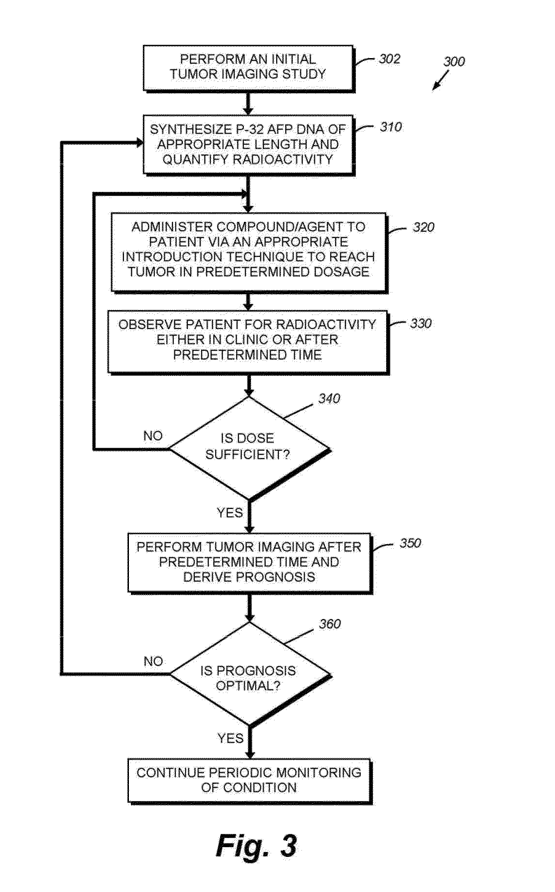

It is contemplated than an appropriate preparation of the above-described compound/agent including P-32 labeled AFP DNA can be employed in the medical treatment of liver cancer, and potentially other forms or cancer, in accordance with an associated treatment protocol. In an illustrative embodiment, the treatment method (as also shown in the flow diagram of FIG. 3) is as follows:

Patients selected for this treatment method (procedure 300 in FIG. 3) are typically those who have been diagnosed with liver cancer, and exhibit an elevated level of AFP. This treatment may better benefit the late stage liver cancer and/or with metastasis which are not amenable for surgery. Contraindication of radioactive therapy should be excluded from this treatment.

Desirably, before treatment begins, the each patient should undergo a tumor imaging study using an appropriate image modality or modalities (e.g. CT Scan, PET Scan, MRI, etc.) (step 302).

Using PCR or DNA synthesis machine to produce the P-32 AFP DNA fragments, the DNA length designed is between 30-2032 bp in an illustrative embodiment. The DNA fragments can be any part of the whole AFP DNA sequence, and it is expressly contemplated that a more-narrow size range can be defined in alternate embodiments. For example, if a certain size range of fragment is shown to more effectively penetrate a cancer cell but not normal cell by experimentation, then that range of fragment sizes is selected for use in the compound. The materials to be used in synthesizing the compound/agent are as described in detail in Sections I-III above (step 310). The radioactivity of the compound/agent is quantified to assist in administering the proper dose.

A predetermined dosage—for example 1 to 160 mci of P-32 AFP DNAs are administered to each patient, illustratively by injecting the compound via the peritoneal cavity or administering it through angioplasty to liver vessels (artery, etc.) to target the tumor (step 320). The amount of administered radioactivity can be varied up and down 200% of the dose range as described above based on patient's age, body weight and the size of tumor. More generally, it is expressly contemplated that a wide variety of drug-delivery techniques and routes of delivery can be employed in any of the embodiments contemplated herein. For example the techniques for administering a predetermined dosage of the compound include, but are not limited to, delivery via (a) oral ingestion, (b) injection into a peritoneal cavity of the human body, (c) intravenously, and (d) injection via a liver artery of the human body, (e) directly to the tumor tissue.

Each patient should be observed, typically in a special nuclear medicine ward (with radioactivity precautions in place) for one to three days, particularly if a higher dose (higher than 60 mci) is administered (step 330). If the dosage is insufficient (decision step 340) after initial observation, further compound/agent can be re-administered in an appropriate dosage (step 320), and the patient is re-observed (step 330). Once the patient has received the appropriate dosage, he or she can be discharged (via decision step 340). Precautions should be provided in detail upon discharge to the patient to avoid contamination of his or her direct environment—especially if small children are present.

A follow-up tumor imaging study is desirably performed one to three months after the treatment (step 350) for comparison with the pre-treatment study (step 302) to determine the effectiveness of the treatment. Based upon this examination, a prognosis can be derived by the practitioner.

Patients may require one or more follow-up treatments (injection of the P-32 DNA fragments and subsequent monitoring according to steps 310-360) to achieve optimal prognosis (decision step 360). When a desirable prognosis is achieved, the patient can be placed upon a less frequent, but still-diligent schedule of observation for recurrence of the condition (step 370).

### V. Use of the Compound in a Diagnostic Procedure

It is also expressly contemplated that the illustrative compound and method can be employed to perform cancer diagnosis. In an illustrative embodiment the following steps are employed:

1. The P-32 labeled DNA fragment is administered to the patient in a manner similar to that described above for treatment. The dosage can vary as described above, potentially being lower, as the compound is being used in a diagnostic context, rather than a treatment context.

2. The patient is observed, potentially in a nuclear medicine ward for between approximately 24-72 hours after which time the compound has sufficiently and selectively bound to genomic DNA in the affected cancer cells. Alternatively, where the dose is sufficiently low, the patient may be released from the clinical environment during the relevant period and return for scanning

3. The patient is subjected to a whole body scan (or a localized scan where appropriate) typically between 24-72 hours after administration of the P-32 DNA fragment compound.

4. A practitioner (radiologist, etc.) studies the results of the scan to study regions where the tumor and/or metastasis exists in the patients body, these regions being highlighted in the scan based upon the P-32 emissions. Thereafter one or more practitioners perform a diagnosis of the studied condition for follow-up treatment.

It should be clear that the compound/agent described herein, as well as the illustrative medical treatment method employing the compound/agent provides a significant tool in the treatment of certain types of cancerous conditions. This method applies treatment selectively, and with minimal risk of over-exposure to radioactive substances.

### VI. Improvements and Further Embodiments

In further embodiments, it is expressly contemplated that a (typically) double-stranded DNA sequence other than the above-described AFP gene and/or another gene can be employed to treat a cancer in accordance with illustrative embodiments herein. The sequence and/or fragment can have a minimum length of approximately 20 and illustratively over 30 bp. More generally, the term “short length” (or alternatively as used herein, “oligonucleotide”) or “long”/“longer”/“large”/“larger” DNA fragment (more than 30 bp″) with reference to a DNA fragment shall refer to a number of base pairs that can be absorbed selectively almost exclusively (so as to not result in healthy cell death from P-32 radiation exposure), or exclusively, by cancer cells. It is expressly contemplated that the techniques described herein can be applied to a variety of cancerous conditions, including those of various organs other than the liver. In general, the binding of DNA with radioisotope (e.g. P-32) to genomic DNA in a cell can occur via the mechanism of recombination, or via other mechanisms. More generally, the P-32 labeled DNA fragment is absorbed by the cancer cells selectively and in a manner that primarily and/or exclusively inflicts cell destruction on the cancerous cells, while leaving the non-cancerous cells relatively unharmed.

According to a further embodiment, DNA for use in the administration of the treatment compound can be provided from the patient's own blood. Illustratively, the blood is drawn from the patient and DNA-containing components are isolated—for example by centrifuge. These components are used to extract and purify any present DNA strands. Such can be accomplished using a known purification protocol, such as the QIAamp Viral Spin Kit. Alternatively, patient's DNA can be purified directly from the patient's tumors or body fluid using a conventional protocol. In a further step, the DNA fragments are prepared and labeled with P-32. This P-32 labeling can be accomplished, illustratively, with random-primer PCR or standard PCR methods with designed primers, as described above and as known in the art. Once the DNA from the patient's blood (venous/arterial or body fluid or tumor-derived) is purified, replicated and labeled, the resulting compound, in solution, is injected into an artery that supplies the tumor using an appropriate intervention method, Alternatively, injection is into one or more appropriate arteries, veins, tumors and/or body cavities of the patient—for example the peritoneal cavity.

In further embodiments, the compounds embodied as DNA fragments labeled with P-32 can be used in combination with trans-artery lipiodol, which serves to maintain the presence of such compounds within the tumors of hepatocellular carcinoma for a longer duration than without (free of) lipiodol. Illustratively, the lipiodol and P-32 labeled DNA fragments can be combined together before injection to a patient, or administered via separate injections in appropriate doses—the DNA fragments being administered in accordance with the dosing principles described herein and the lipiodol being administered in levels known to those of skill—for example 2 ml of the compound emulfcified with 3 ml lipiodol then injected into the artery which supplies a liver cancer.

Illustratively, before treatment, during treatment and after treatment, ethylenediamine tetra-acetic acid (EDTA), in the form of disodium-EDTA, can be administered intravenously, or directly into a liver artery. When administered intravenously, the goal is to infuse the prescribed dose of disodium-EDTA into the patient slowly over 3 to 6 hours, with a maximum rate of infusion of approximately 16 mg/min. The purpose is to suppress DNA exonucleases and endonucleases, which can break down the compounds in blood and tissues

In various embodiments, based upon experimental data it is contemplated that a P-32 labeled DNA fragment for cancer treatment in the range of 30 bp, up to approximately 10 kbp (10,000 bp) can be implemented. Fluorescent studies have revealed that, when larger DNA fragment is introduced to liver cancer cells, fluoresce occurred mainly on the membrane of the cells, which suggests the cancer cells' uptake of the compounds (larger DNA fragment compared with oligoneucleotides) is likely though endocytosis. Normal liver cells lack of endocytosis, which can suggest their general inability to uptake larger DNA fragments. While oligonucleotides have been tested and used for treatment/diagnosis of different diseases, such are generally in the size range of approximately 20 bp and serve as antisense drugs. Notably, both normal cells and cancer cells can uptake such oligonucleotides. The illustrative compound and method described herein uniquely employs comparably “large” DNA fragments to treat a disease—particularly cancer. With the selective uptake by cancer cells, this large DNA fragment can also be used for drug delivery; for example the known cancer drugs can be conjugated to the DNA fragment with a designed length then applied to patients to have better targeting and fewer negative side effects. For example; the cancer drug Fluorouracil (5-FU) can be conjugated to a long/large DNA fragment (57 bp), then inject intravenously to treat colon cancer to achieve better selectivity.

In various embodiments, for stability (resistance to be broken down by nucleases in vivo), the DNA fragment can be modified to phosphorothioate end by using phosphorthioate PCR primers. Phosphorothioates (or S-oligos) are a variant of normal DNA in which one of the nonbridging oxygen atoms is replaced by a sulfur atom. The sulfurization of the internucleotide bond substantially reduces the action of endonucleases and exonucleases. Phosphorothioates can be synthesized, for example, by two principal routes: by the action of a solution of elemental sulfur in carbon disulfide on a hydrogen phosphonate, or by the more recent method of sulfurizing phosphite triesters with either tetraethylthiuram disulfide (TETD) or 3H-1,2-bensodithiol-3-one 1,1-dioxide (BDTD). Illustratively phosphrothioates PCR primers can be used to generate the P-32 labeled DNA fragment via the same PCR method as described further above. The illustrative primers are commercially available as customized DNA from companies such as the Sigma-Aldrich Corporation of Saint Louis, Mo.

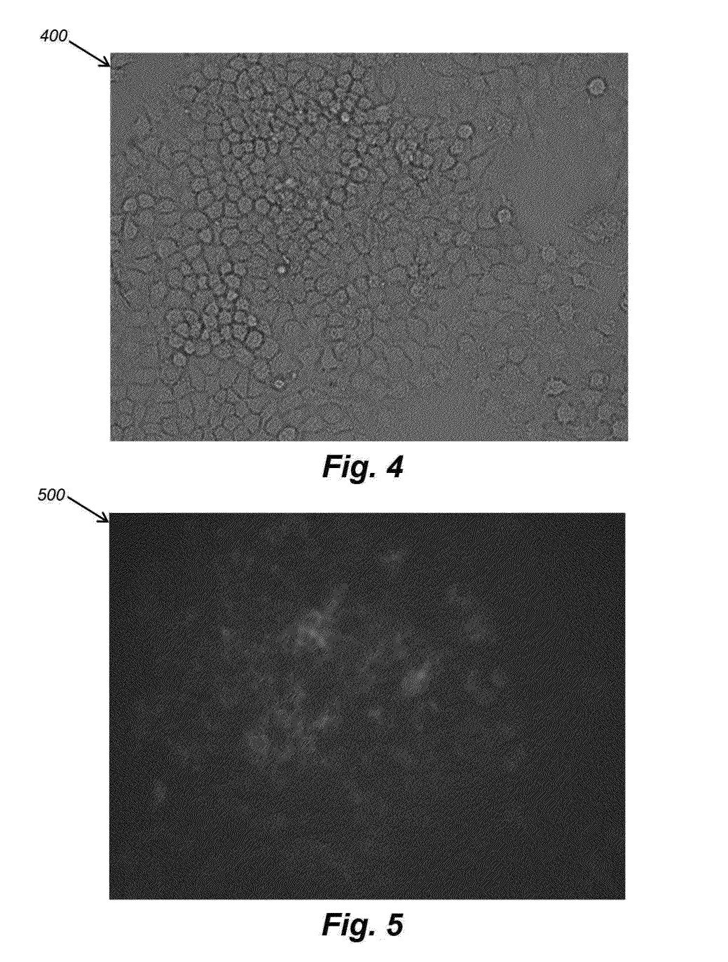

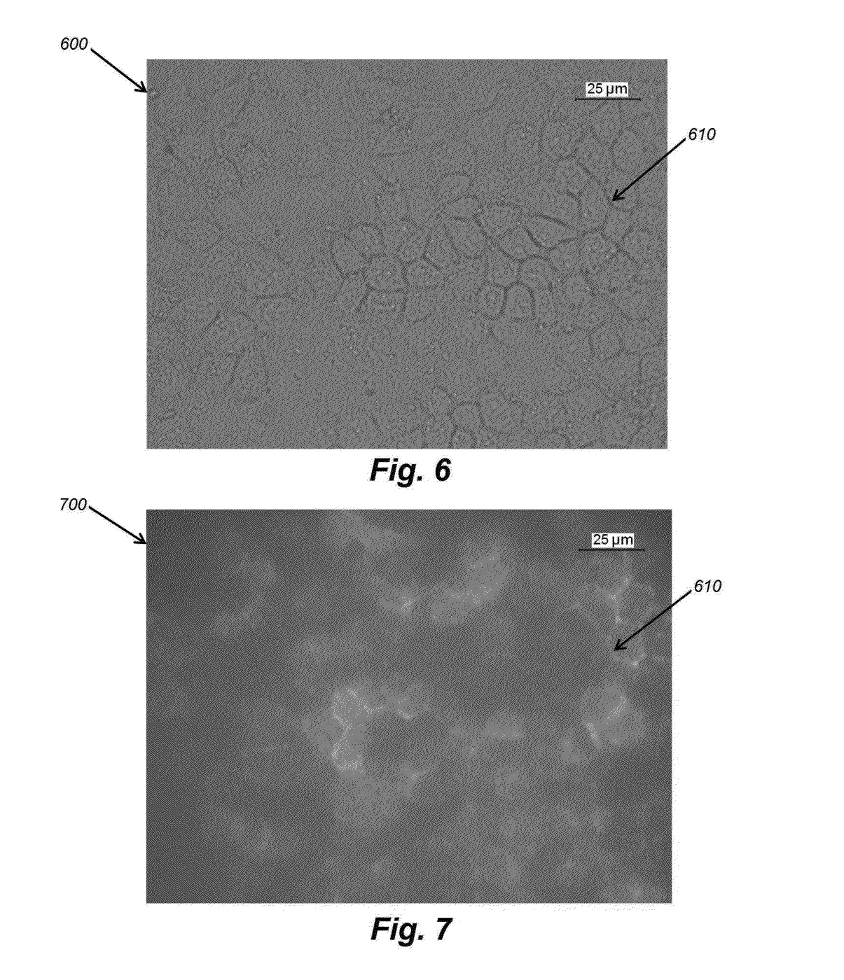

Reference is now made to FIGS. 4-7, which provide images of Huh7 cells incubated overnight. A 1.2 kb human-AFP DNA was labeled with fluorescence using a PCR method and incubated with Huh7 cells overnight. In FIG. 4, the cell sample 400 is depicted with membranes appearing as darker lines. A fluorescent image 500 of the cell sample 400 is shown in FIG. 5. Notably, the majority of fluorescence appears within the membrane region, as the fluorescent staining appears to be primarily located on the cell membranes. A close up of a region of the sample 600 is shown in FIG. 6 and a particular membrane 610 is denoted on the image. In the fluorescent view 700 of the sample (FIG. 7), the same membrane 610 appears brightly delineated. This pattern suggests that the large DNA uptake by the cancer cells occurs via a different pathway or process than that of oligonucleotide in which the fluorescence is located in the cytoplasm and nuclei of the cell sample. Based on the location, the uptake of large DNA by cancer cells likely occurs through endocytosis, which does not occur in normal liver cells.

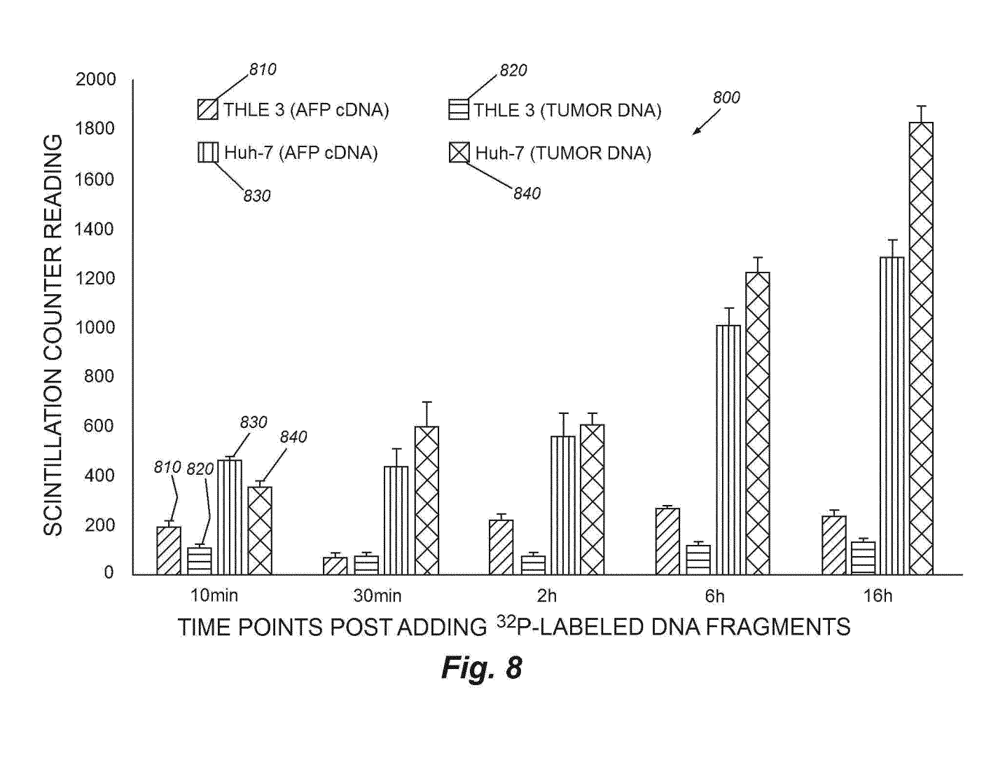

Reference is now made to FIG. 8, which shows a graph 800 describing the results of an uptake test using compounds in accordance with embodiments herein. The graph plots scintillation counter readings within a sample versus time for application two different P-32 labeled compounds is two respective cell types (normal and cancerous): THLE 3 (applying AFPc DNA) 810, THLE 3 (applying Tumor DNA) 820, Huh7 (applying AFP cDNA) 830, Huh7 (applying Tumor DNA) 840. As shown, the uptake of P-32 labeled AFP or tumor genomic DNA fragments by cancer cells (Huh7), which express a high level of endogenous AFP gene exhibit a high ability of uptake of both DNA fragments. This provides a possible suggestion that the uptake of specific types of DNA by cancer cells is specifically based on the need for such by the cells. As noted, this graph 800 also compares the uptake efficiency of P-32 labeled AFP versus genomic DNA fragments by Huh7 cells at different time points (e.g. 10 minutes, 30 minutes, 2 hours, 6 hours, and 16 hours). The results indicate that Huh7 cells can uptake both AFP and genomic DNA fragments at a comparatively similar efficiency. However, the uptake efficiency of genomic DNA is slightly better. In all instances, uptake by the cancerous cells is significantly greater over time than by the non-cancerous cells. This, again, supports the use of compounds and treatment methods described herein in the selective lethal irradiation of cancer cells whilst allowing normal cells to avoid such lethal dosage.

Note again that the DNA described in accordance with various embodiments are considered “large” DNA fragments as compared with oligonucleotides and similar structures. From experimental results, the large DNA fragment can be taken up by cancer cells is believed to be due to the cancer cells evolve the capacity of endocytosis—which does not occur in normal organ cells. This difference enables the selectivity of the compounds. In a human treatment method, the tumor is initially imaged to determine tumor size and characteristics.

According to further embodiments, the following is a description of five compounds that can be employed according to the teachings herein as treatments for liver cancer (and potentially other cancers and cancerous conditions, in which uptake of the compounds occurs selectively in cancer cells). These compounds are synthesized/generated using the PCR labeling techniques described above. The primers are listed below, and are ordered as modified DNA of Phosphorothioates. The PCR template DNA is the same sequence provided hereinabove.

Compound 1 (57 bp)

P1-57-S (1258-1337): TGCGTTTCTCGTTGCTTACAC
P2-57-AS (1354-1373): ATCAGCTCCGACGAGGTC

Compound 2 (150 bp)

P1-150-S (1501-1522): ACTCCAGTAAACCCTGGTGTTG
P2-150-AS (1630-1653): CTGAGCTTGGCACAGATCCTTATG

Compound 3 (310 bp)

P1-150-S (1501-1522): ACTCCAGTAAACCCTGGTGTTG
P2-310-AS (1786-1820): CAGCAAAGCAGACTTCCTGTTCCTG

Compound 4 (570 bp)

P1-570-S (1241-1263): ACATCCAGGAGAGCCAAGCATTG
P2-310-AS (1786-1820): CAGCAAAGCAGACTTCCTGTTCCTG

Compound 5 (1620 bp)

P1-1620-S (22-48): CTGGCAACCATGAAGTGGGTGGAATCA
P2-150-AS (1630-1653): CTGAGCTTGGCACAGATCCTTATG

It should be clear that the use of P-32 labeled DNA fragments has clear and proven advantages in the selective irradiation/killing of certain cancerous cells via selective uptake versus that observed in normal cells. The fragments can comprise a known gene, such as the AFP gene or a genomic tumor DNA. Illustratively, the primers used herein can be modified DNA of Phosphorothioates, and fragments in a range of 30 bp, up to approximately 10 kbp (10,000 bp) can be employed.

The foregoing has been a detailed description of illustrative embodiments of the invention. Various modifications and additions can be made without departing from the spirit and scope of this invention. Each of the various embodiments described above may be combined with other described embodiments in order to provide multiple features. Furthermore, while the foregoing describes a number of separate embodiments of the apparatus and method of the present invention, what has been described herein is merely illustrative of the application of the principles of the present invention. For example, the size of the AFP fragment employed is highly variable, as is the portion of the overall fragment being employed for binding with P-32. The use of an AFP fragment is only one example of possible DNA strands that can be bound with P-32 for injection into cancerous tissue. It is contemplated that the medical treatment method herein can employ other types of DNA fragments that are shown to be associated with certain types of cancer cells. For example, the same methods described herein can be employed to label a calcitonin DNA fragment with P-32 to treat medullary thyroid carcinoma. Likewise a combination of different types of P-32 labeled fragments can be employed in a single injection to selectively bind with different portions of genes or different cell types in a tumor mass. Moreover, the use of the illustrative compound/agent in a treatment protocol can be supplemented with other forms of conventional treatment, such as chemotherapy, radiation, and the like, if needed to achieve the most desirable prognosis. Accordingly, this description is meant to be taken only by way of example, and not to otherwise limit the scope of this invention.

What is claimed is:

1. Compound for treatment of cancerous tumors comprising:
DNA fragments labeled with P-32, the DNAs being capable of selectively taken up by predetermined cancerous cells within the tumor to bind with genomic DNA within the cancerous cells.

2. The compound as set forth in claim 1 wherein the DNA fragment comprises an AFP gene fragment.

3. The compound as set forth in claim 1 wherein the DNA fragment is defined by a length of between 30 and approximately 10,000 base pairs (bp).

4. The compound as set forth in claim 1 wherein the DNA fragment comprises a sequence that is adapted to bind through at least one of recombination and another binding mechanism to an associated sequence in genomic DNA that is prevalent in a predetermined type of cancer cell.

5. The compound as set forth in claim 1 wherein the DNA fragment is prepared into a solution that enable injection into a human body.

6. The compound as set forth in claim 5 wherein the solution provides a radioactivity of between approximately 1 mci and 160 mci of radiation to the human body.

7. The compound as set forth in claim 5 wherein the solution is prepared for administration to a liver tumor.

8. The compound as set forth in claim 5 wherein the solution is prepared for injection into at least one of a vein, artery, body cavity and directly into tumor.

9. The compound as set forth in claim 1 wherein the DNA fragment is derived from blood, body fluid or a tumor of a patient or lab produced.

10. The compound as set forth in claim 1 wherein primers used to generate the DNA fragment are modified DNA of phosphorothioates.

11. The compound as set forth in claim 10 wherein the primers (P1 and P2) include at least one of the following gene sequences;
(a) P1-57-S (1258-1337): TGCGTTTCTCGTTGCTTACAC
P2-57-AS (1354-1373): ATCAGCTCCGACGAGGTC,
(b) P1-150-S (1501-1522): ACTCCAGTAAACCCTGGTGTTG
P2-150-AS (1630-1653): CTGAGCTTGGCACAGATCCTTATG,
(c) P1-150-S (1501-1522): ACTCCAGTAAACCCTGGTGTTG
P2-310-AS (1786-1820): CAGCAAAGCAGACTTCCTGTTCCTG,
(d) P1-570-S (1241-1263): ACATCCAGGAGAGCCAAGCATTG
P2-310-AS (1786-1820): CAGCAAAGCAGACTTCCTGTTCCTG,
and
(e) P1-1620-S (22-48): CTGGCAACCATGAAGTGGGTGGAATCA
P2-150-AS (1630-1653): CTGAGCTTGGCACAGATCCTTATG.

12. A kit containing instructions for use of the compound as set forth in claim 1.

13. A medical treatment method for a tumor in a human body comprising the steps of:
determining a condition of a tumor in the human body;
synthesizing a compound containing a DNA fragment labeled with P-32, the DNA being capable of penetrating predetermined cancerous cells within the tumor to bind with genomic DNA within the cancerous cells;
administering the compound so as to be delivered to the cancerous cells in a predetermined dosage;
monitoring the delivered dosage and repeating the step of administering as required; and
re-determining the condition of the tumor after at least one step of administering to provide a prognosis.

14. The medical treatment method as set forth in claim 13 wherein the DNA fragment is derived from at least one of a DNA sequence and tumor genomic DNA.

15. The medical treatment method as set forth in claim 13 wherein the predetermined dosage is between approximately 1 and 160 mci of radiation delivered.

16. The medical treatment method as set forth in claim 13 wherein the step of administering includes delivering the predetermined dosage of the compound through at least one of (a) oral ingestion, (b) via a peritoneal cavity of the human body, (c) intravenously, (d) via an artery supplying the tumor, and via direct injection to the tumor.

17. The medical treatment method as set forth in claim 13 wherein the DNA fragment is a length of between 30 bp and 10 kbp.

18. A method for diagnosing a tumor in a human body comprising the steps of: synthesizing a compound containing a DNA fragment labeled with P-32, the DNA fragment being capable of penetrating predetermined cancerous cells within the tumor to bind with genomic DNA within the cancerous cells;
administering the compound so as to be delivered to the cancerous cells in a predetermined dosage;
performing a scan of at least a portion of the human body so as to locate regions that contain the P-32 in bound form to the genomic DNA, the predetermined dosage being sufficient to provide indication of the regions under the scan; and
reviewing results of the scan and diagnosing at least one of a tumorous condition and metastasis based upon the review.

19. The method as set forth in claim 18 wherein the DNA fragment is derived from at least one of a DNA sequence and tumor genomic DNA fragment.

20. The method as set forth in claim 18 wherein the step of administering includes delivering the predetermined dosage of the compound through at least one of (a) oral ingestion, (b) via a peritoneal cavity of the human body, (c) intravenously, (d) via an artery supplying the tumor, and (e) via direct injection into the tumor.

21. The method as set forth in claim 18 wherein the DNA fragment comprises a sequence that is adapted to bind through at least one of recombination and another mechanism, to an associated gene in genomic DNA that is prevalent in a predetermined type of cancer cell
